(19) United States
(12) Patent Application Publication
(10) Pub. No.: US 2020/0046874 A1
(43) Pub. Date: Feb. 13, 2020

(54) COMPOSITIONS FOR TISSUE HEMOSTASIS, REPAIR AND RECONSTRUCTION

(71) Applicant: Abyrx, Inc., Irvington, NY (US)

(72) Inventors: Richard L. KRONENTHAL, Irvington, NY (US); Aniq DARR, Irvington, NY (US); John PACIFICO, Irvington, NY (US)

(21) Appl. No.: 16/343,604

(22) PCT Filed: Oct. 20, 2017
(86) PCT No.: PCT/US2017/057548

§ 371 Date: Apr. 19, 2019

Related U.S. Application Data
(60) Provisional application No. 62/410,464, filed on Oct. 20, 2016.

(51) Int. Cl. A61L 24/00; A61L 24/04

(52) U.S. Cl. CPC A61L 24/0036; A61L 2430/02; A61L 2400/04; A61L 24/043

(57) ABSTRACT

The disclosure provides settable surgical materials that are useful, for example, as cement and/or adhesive compositions or as tissue void or space fillers. The invention also provides related compositions, including surgical kits and packages, as well as methods of making and using the settable surgical materials described here.

### CROSS-REFERENCE TO RELATED APPLICATIONS

This application is a National Stage Application, filed under 35 U.S.C. 371, of International Application No. PCT/US2017/057548, filed on Oct. 20, 2017, which claims priority to U.S. Provisional Patent Application No. 62/410,464, filed on Oct. 20, 2016, the contents of each of which are herein incorporated by reference in their entirety.

### FIELD OF THE INVENTION

The present invention relates to the field of medical implant compositions for use in tissue hemostasis, repair and reconstruction.

### BACKGROUND

Biodegradable polymers have become increasingly important for a variety of biomedical applications including biomedical implants, such as stents, and coatings applied to those implants, tissue engineering scaffolds, and soft-tissue adhesives. Segmented polyurethane elastomers and other biocompatible materials in particular have come into wide use as biomaterials due to their mechanical properties and chemical versatility. PCT International Application Publication No. WO 2004009227 describes certain degradable polyurethane compositions for use as tissue engineering scaffolds. U.S. Pat. No. 6,306,177 (Felt et al.) describes curable polyurethane compositions comprising a plurality of parts capable of being sterilized, stably stored, and mixed at the time of use in order to provide a flowable composition upon mixing that is sufficiently flowable to permit it to be delivered to the body by minimally invasive means. U.S. Patent Application Publication No. 20050013793 (Beckman et al.) also describes degradable polyurethanes e.g., for tissue engineering and particularly for bone repair and replacement. U.S. Pat. No. 4,829,099 (Fuller et al.) describes certain absorbable polyisocyanates for use as surgical adhesives. U.S. Pat. Nos. 8,002,843 and 7,985,414 (Knaack et al.) describe biodegradable polyisocyantes (such as lysine diisocyanate) with an optionally hydroxylated biomolecule to form a degradable polyurethane. U.S. Pat. No. 7,964,207 (Deslaurier, et al.) describes osteoconductive polyurethane compositions having mechanical properties consistent for use in bone repair.

For certain applications, in addition to being biodegradable, it is advantageous for a surgical implant to be moldable or formable, for example, to optimize its placement at the implant site and/or to fill voids in hard or soft tissue at the site of implantation. U.S. Pat. Nos. 8,431,147 and 8,282,953 (Warsaw Orthopedics, Inc.) describe malleable implants containing demineralized bone matrix. The “malleable implant compositions” described in these patents contain a particulate solid collagen material and a particulate solid DBM material along with a liquid carrier that comprises an aqueous gel of alginate. Alginate/DBM based compositions also are described in U.S. Pat. No. 8,506,983 (Warsaw Orthopedic, Inc). US 2013/0236513 (Guelcher et al, Vanderbilt Univ.) describe polyurethane composites that, in some aspects, may be “processed” as reactive liquids that subsequently cures in situ to form a solid composite.

Many non-isocyanate-based surgical adhesives and cements have also been developed. For example, Stewart, et al., WO 2009094060 and U.S. Pat. No. 8,283,384, describe complex adhesive coacervates composed of mixtures of one or more polycations, one or more polyanions and one or more polyvalent metallic cations. The polycations and polyanions are crosslinkable with one another upon curing. Covalent crosslinks may be derived from electrophilic and nucleophilic group interactions on each polymer chain. Crosslinkable electrophilic groups may be, e.g., esters, ketones, lactams, lactones, isocyanates and aldehydes while crosslinkable nucleophilic groups include, e.g., aromatic polyhydroxy groups, e.g., DOPA, that are oxidizable to quinoidal structures. Such coacervates are said to be particularly useful adhesives for under-water applications such as physiological conditions involving moist or wet soft or hard tissues.

Coacervates in the form of liposomes can be lyophilized and functionally reconstituted. Cavallo et al. (WO 1998/036736), has reported that if trehalose, a biocompatible cryoprotective glucose derivative, is added to a liposome prior to lyophilization, the recovery of intact, functional liposomes is improved after the freeze-dried product is reconstituted with body fluids or saline.

One disadvantage of coacervate-based settable adhesives is that they are derived from aqueous solutions and, as such, cannot have the intrinsic strength associated with neat, undiluted materials such as, for example, those based on polyurethanes, polymethylmethacrylates, polyvinylidenemalonate esters, epoxide resins and polycyanoacrylates, none of which conventionally involve dilutive vehicles.

Another approach to non-isocyanate settable surgical adhesives and cements is described by Hess in US 2011/0277931. This application describes phosphoserine and similar compounds, in combination with calcium phosphate-containing, e.g., calcium phosphate or tetracalcium phosphate cements. These are said to have improved properties and form non-crosslinked interpenetrating networks in the presence of a polymer, e.g., polyglycolide or poly(vinylpyrrolidone) that contains either an electronegative carbonyl oxygen atom of an ester group or an electronegative nitrogen atom of an amine group as bonding sites on the polymer surface to available multivalent metal ions. One disadvantage of this approach is that the absorbable polyesters are neither stable nor soluble in aqueous vehicles. Another problem is that the initially-adhesive cement is derived from a solution or suspension vehicle and will not provide the bonding strength of neat, undiluted adhesive or cement systems. Further, US 2011/0277931 teaches the inconvenient mixing of several solid materials with an aqueous vehicle, the mixture curing into a cement after application to tissue.

Another approach to non-isocyanate surgical adhesives and cements is described in WO 2011/075580. This application describes a bone cement comprised of two precursor pastes. The first precursor acidic, aqueous paste comprises at least one acidic calcium phosphate mineral, e.g., monocalcium phosphate monohydrate, at least one synthetic polymer-based paste stabilizing agent, e.g., polyvinylpyrrolidone, an acidic pH buffering agent, e.g., citric acid and a humectant, e.g., glycerol. A second precursor paste comprises an alkaline, non-aqueous paste with at least one basic calcium phosphate mineral, e.g., tetracalcium phosphate, at least one paste-stabilizing agent, e.g., polyethylene glycol, an antioxidant, e.g., thioglycerol, a surfactant, e.g., polysorbate 80 and a solvent, e.g., propylene glycol. When the precursor acidic aqueous and alkaline non-aqueous pastes are mixed, a rapidly setting, non-adhesive calcium phosphate bone cement forms. Disadvantages of the bone cement described in WO 2011/075580 include time-consuming manual mixing of the two pastes is required just before using. If using a static mixing device in conjunction with syringe delivery, it is unlikely that two viscous pastes, one aqueous and one non-aqueous, can be adequately mixed. The system described provides a cement rather than a settable adhesive.

Other cements used for securing implants, such as metallic artificial hip prostheses, into bone are usually prepared just before use in the operating room by mixing one or more liquid acrylate and/or methacrylate monomers and preformed polyacrylate and/or polymethacrylate polymers with a polymerization catalyst that generates free radicals or ions which induces polymerization of the monomers. This system, while effective in generating clinically acceptable cements, has drawbacks such as unpleasant monomer odor, heat generation (exotherm) during polymerization that may thermally destroy surrounding tissue and monomer toxicity, often manifested by a reduction in the patient's blood pressure.

There is a need for improved surgical cement and/or adhesive materials that exhibit low, physiologically acceptable polymerization exotherms, have monomer components of low toxicity and vapor pressure, are easily prepared in the operating room, and are compatible with antimicrobials and other drug substances.

### SUMMARY OF THE INVENTION

The compositions described herein are settable surgical materials that are useful, for example, as cement and/or adhesive compositions. The settable surgical materials described here may also be used as tissue void or space fillers. The invention also provides related compositions, including surgical kits and packages, as well as methods of making and using the settable surgical materials described herein. The terms settable and curable are used interchangeably herein.

In embodiments, the settable compositions described herein consist of at least two separate and individual reactive putty components. Each separate putty of the at least two separate and individual reactive putty components is reactive with component(s) in the other putty or putties of the settable composition. Thus, each separate putty contains agents which react with agents in the other putty or putties of the composition when the separate putties are combined. The combination of the individual reactive putties, for example by mixing or kneading, forms a single, substantially homogenous material that cures over a period of time into a final hardened form. Therefore, in embodiments, each component of a settable composition of the invention is in the form of a putty and the single putty that results from their combination is also in the form of a putty for a period of time after their combination, which combination effects initiation of the curing reaction. The term “putty” in this context refers to a composition that is soft, moldable, optionally non-elastic, and cohesive.

In another embodiment, the disclosure provides settable compositions that are not in the form of reactive putties but instead are comprised of compressed stacks of dry, potentially inter-reactive lyophilized sponges, particles or films, and combinations thereof. In embodiments, at least two layers of the compressed stack each contain separate anionically- and cationically-charged polymers, each containing an optional biocompatible cryoprotective agent. In embodiments, an optional third layer of the compressed stack contains a metallic polyvalent salt. When this construct is hydrated, for example by placing it at the surgical site, an adhesive, settable coacervate is formed.

The disclosure also provides a multi-putty settable surgical adhesive composition comprising a first putty comprising a suspension of particles from a ground lyophilized sponge containing a polyanionic polymer salt and a separate second putty comprising a suspension of particles from a ground lyophilized sponge containing a polycationic salt wherein, when the two putties are combined together with a polyvalent metallic salt, they form an adhesive coacervate that cures into a hardened state.

The disclosure also provides a curable surgical adhesive composition comprising two separate individual reactive putties, wherein Putty A comprises a mixture of PEG monostearate and PEG containing tetracalcium phosphate, phosphoserine and dried buffer-producing powder and Putty B comprises a mixture of PEG monostearate and. PEG containing fibrous or powdered absorbable polymer or fibrous or powdered nonabsorbable polymers containing, e.g., ester linkages.

The disclosure also provides an initially adhesive bone cement formed by kneading together two or more putties wherein the acidic first putty comprises an alkylene oxide glycol mono-fatty acid ester, an alkylene oxide glycol, an acidic phosphate salt, an acidic pH buffering agent, an optional humectant and an optional poloxamer and the basic second putty comprises an alkylene oxide glycol mono fatty acid ester, an alkylene oxide glycol, a basic calcium phosphate salt, a surfactant and, optionally, osteoconductive and/or osteoinductive agents.

The disclosure also provides an initially adhesive bone cement, formed by kneading together two or more putties wherein the first putty is comprised of one or more basic calcium phosphate salts, a polyol, a polyol-terminated polyurethane prepolymer and an optional osteoactive ceramic and the second putty is comprised of an acidic calcium phosphate salt, a polyisocyanate, an isocyanate-terminated prepolymer, and an optional osteoactive ceramic.

The disclosure also provides an initially adhesive bone cement formed by kneading together two or more putties wherein the first putty is comprised of an aliphatic or aromatic polyisocyanate, an osteoactive calcium phosphate, magnesium oxide, dibasic potassium phosphate, tetracalcium phosphate and a viscous isocyanate-terminated polyurethane prepolymer and the second putty is comprised of phosphoserine, one or more polyols, BMP and/or DBM, optionally an N-alkyl pyrrolidone, an osteoactive calcium phosphate and a viscous, hydroxyl-terminated polyurethane prepolymer.

In accordance with any of the embodiments described here, the surgical compositions are sterile or sterilizable.

### DETAILED DESCRIPTION OF THE INVENTION

The embodiments described here provide settable surgical compositions for use as adhesives, cements, tissue void fillers, etc., that are in an advantageous solid form, either in the form of a solid structure (such as a compressed stack of layers of a solid material, or a non-woven fabric) or in the form of a set of separate, individual reactive putties. The solid form of the present compositions provides improvements in ease of use and handling compared to existing materials for forming adhesive and/or settable surgical compositions. In this context the term a ‘solid’ form is meant to distinguish from a liquid form, meaning that the present solid compositions retain a definite shape, while still being moldable in some embodiments and/or for a period of time before complete cure has been achieved, and do not flow perceptibly under moderate stress at room or body temperature.

In embodiments, the present disclosure provides solid dry adhesive compositions in the form of a compressed stack of layers consisting of a lyophilized sponge material, described in more detail below.

In embodiments, the present disclosure also provides unique multi-putty settable surgical compositions. The multi-putty settable surgical compositions each comprise at least two separate, individual reactive putties that are combined (e.g., by hand kneading or mixing, which mixing may also be assisted by a mechanical device) just prior to use (e.g., just prior to application to tissue) into a single, substantially homogenous putty. The resulting putty is applied to the surgical site where it sets over a period of time, typically from an initially adhesive phase to a hardened form that is suitable for various surgical applications such as a bone or tissue adhesive, cement, or bone void filler. This putty that is formed from the combination of two or more separate reactive putties and applied to the surgical site may also be referred to herein as the “implant”. The implant may be body-absorbable or non-absorbable, as described below. Thus, the multi-putty settable surgical compositions described here are ‘sellable’ in the sense that they will set following their combination into a single putty (the implant).

In one embodiment, there is described a multi-putty adhesive composition comprising two separate, individual reactive putties, one putty comprising a suspension of particles from a ground lyophilized sponge containing a polyanionic polymer salt and the second putty comprising a suspension of particles from a ground, lyophilized sponge containing a polycationic polymer salt. This and related embodiments are described in more detail below under the heading “Multi-Putty Settable Adhesives Using Lyophilized Sponge-Based Materials”.

In another embodiment, there is described a multi-putty adhesive composition based on polyethylene glycol (“PEG”) and fatty acid esters of PEG. In embodiments, a PEG ester based adhesive composition comprises two separate, individual reactive putties, one putty comprising a mixture of polyethylene glycol (“PEG”) monostearate and PEG containing tetracalcium phosphate, phosphoserine and dried buffer-producing powder, and the second putty comprising a mixture of PEG monostearate and PEG containing fibrous or powdered absorbable polymers or fibrous or powdered non-absorbable polymers, each containing ester linkages in the backbones. This and related embodiments are described in more detail below under the heading “Multi-Putty Settable Surgical Materials Based on Polyethylene glycol (“PEG”) and Fatty Acid Esters of PEG”.

In another embodiment, there is described a multi-putty settable composition based on poly (methyl methacrylate) (PMMA). In embodiments, the PMMA-based composition comprises two separate, individual reactive putties, one putty comprising liquid acrylate and methacrylate monomers and powdered polyacrylate and/or polymethacrylate polymers, suspended or dissolved in an inert liquid vehicle, and the second putty comprising free radical or ionic polymerization initiator sources and powdered polyacrylate and/or polymethacrylate polymers suspended or dissolved in an inert liquid vehicle. These embodiments are described in more detail below under the heading “Multi-Putty Cements Based on Poly (methyl methacrylate) (PMMA).

In embodiments, the surgical adhesive compositions described here consist of at least two separate, individual reactive putties. The sum of the two or more separate, individual reactive putties that together form a settable surgical adhesive composition described here may be referred to as a “set” of putties. In all cases, the set of putties comprise reactive components such that when the separate, individual putties of the set are physically combined, the reactive components of each putty in the set react to initiate cure. Thus, the multi-putty settable surgical adhesive compositions described here cure into a final hardened composition following the combination of the set of individual putties of the composition. In embodiments, the individual putties are combined by hand-mixing or kneading the individual putties of a set into a homogenous mass. In embodiments, the individual putties are combined by mechanical or electromechanical means into a homogenous mass. In embodiments, the mechanical or electromechanical means is a modified syringe-like device.

In embodiments, the homogenous mass cures into a final hardened composition at, e.g., room temperature or body temperature. In embodiments, cure occurs without the need to apply additional external heat in excess of the ambient heat of the room (about 24-26° C.) or the heat of the human body (about 37° C.). In embodiments, the period of time for complete cure into a hardened final form is from about 6 to 12 hours or from about 6 to 24 hours. In embodiments, the fully cured composition is drillable or machinable.

The multi-putty surgical adhesive compositions described here are in the form of a set of at least two separate, individual reactive putties. The putty form is distinguishable from a “paste” form. A paste is defined as a thick, viscous fluid. In contrast, a putty is a material exhibiting high plasticity which is capable of being molded and retaining the shape imparted by molding pressure deformation. There are surgical advantages for an orthopedic implant to be putty-like rather than a viscous liquid with paste-like consistency, e.g., visualize paste-like cosmetic cold cream vs. putty-like modeling clay. Such advantages include handling ease, retention of molded shape, adherence to tissue, and minimal adherence to gloves or instruments during use.

In various embodiments described herein, a putty is formed using a suspension or dispersion of particulates within a liquid phase. As an illustrative example of this general form, one can consider the non-medical putty composition referred to as glazier's putty, which is diatomaceous earth or clay suspended in a drying oil such as linseed oil. In embodiments, the particulate material is present in an amount of from about 10% to about 20% by weight of the composition, or from about 20% to 30% , about 30% to 40% , about 40% to 50%, about 50% to 60% , about 60% to 70% or about 70% to 80% by weight of the composition. In embodiments, the particulate material is present in an amount of from about more than 20%, more than 30%, more than 40%, more than 50%, more than 60%, or more than 70% by weight of the composition.

In embodiments, the liquid component in which the particulate material is dispersed or suspended may be a reactive liquid, for example, liquid acrylate and methacrylate monomers, a liquid polyisocyanate or polyisocyanate terminated prepolymer, or a liquid epoxy resin such as triglycidal-p-aminophenol.

In embodiments, the liquid component in which the particulate material is dispersed or suspended may be a non-reactive, nontoxic liquid, referred to herein as an “inert dispersant”. In embodiments, the inert dispersant is as an ester, such as ethyl acetate or tocopheryl acetate, an ether, such as polyethylene glycol or a polxamer (PLURONIC™), or a hydrocarbon, such as decane or toluene, and mixtures thereof. In embodiments, the inert dispersant is a viscous aqueous buffer, for example, an aqueous solution containing a thickening agent such as hypmmellose (hydroxypropylmethylcellulose), sodium carboxymethyl cellulose, sodium carboxymethyl starch, polyvinylpyrrolidone, and mixtures thereof. In embodiments, the inert dispersant may comprise, for example, a poly(alkyleneoxide) glycol or its mono or diester, glycerol, a poloxamer, and mixtures thereof. In embodiments, the inert dispersant is polyethylene glycol monostearate.

The particulate material which is suspended or dispersed in the liquid vehicle may be selected from, for example, one or more of calcium phosphate, siliconized calcium phosphate, substituted calcium phosphates where the substitution is with magnesium, strontium, or silicate, for example, calcium phosphosilicate, calcium pyrophosphate, hydroxyapatite, polyaryletherketone-based materials, polyurethanes, polyureaurethanes, polyureas, fiberglass, synthetic absorbable polymers (e.g., PGA, PLA and their copolymers), polydioxanone, polymethylmethacrylate (PMMA), silicone polymers, glass-ionomer, absorbable phosphate glass, calcium sulfate, tricalcium phosphate (e.g., beta tricalcium phosphate), partially or fully demineralized bone matrix (DBM), or mineralized bone, or any combination of the foregoing. In embodiments, the particulate material which is suspended or dispersed in the liquid vehicle may itself be reactive, for example with the components of the other putty in a two-putty pair, or with the components of another putty in the multi-putty embodiment. In embodiments, the reactive particulate material is a mixture of powdered polyacrylate and polymethacrylate polymers. In embodiments, the particulate material may be selected from one or more of oxidized cellulose, chitosan, collagen, nonabsorbable synthetic polymers such as polytetrafluoroethylene and polyethyleneterephthalate, powdered metals such as titanium and steel, natural fibers such as silk and cotton.

In accordance with any of the multi-putty embodiments described herein, one or more of the separate, individual reactive putties may also contain one or more optional additives. The optional additive is preferably non-reactive with the chemically reactive putty components. In embodiments, the one or more optional additives comprise particulate materials. In embodiments, the particulate material is an osteoconductive material. In some embodiments, the particulate material is osteoinductive and supports or promotes the growth of bone at the application site. In one embodiment, the particulate material is non-resorbable. In certain embodiments, the mean particle size of the optional particulate material is in the micron or submicron range, e.g., nanoparticles. In one embodiment, the mean particle size is from about 0.001 to 0.100 microns, from about 0.100 to 5 microns, from about 5 to 100 microns, from about 5 to 500 microns, or from about 500 to 2000 microns.

In embodiments, the optional particulate material is a carbonate or bicarbonate material. In one embodiment, the carbonate or bicarbonate material comprises or consists of one or more of calcium carbonate, magnesium carbonate, aluminum carbonate, iron carbonate, zinc carbonate, calcium bicarbonate, and sodium bicarbonate. In one embodiment, the optional particulate material comprises or consists of a bone-derived substance (e.g., demineralized bone, bone morphogenetic protein, allograft bone, and/or autogenous bone), calcium phosphate, siliconized calcium phosphate, substituted calcium phosphates (e.g., with magnesium, strontium, or silicate), calcium pyrophosphate, hydroxyapatite, poly(methyl-methacrylate), glass-ionomer, absorbable phosphate glass, calcium sulfate, tricalcium phosphate (e.g., beta tricalcium phosphate), or any combination of the foregoing. Other examples include one or more poly ether ether ketones (e.g., PEEK), REPLACE (Cortek, Inc.), EXPANCEL (Akzo Nobel). In other embodiments, the particulate material is a ceramic such as substituted calcium phosphates (e.g., silicate, strontium or magnesium substitution) or a glass such as bioglass. In one embodiment, the particulate material comprises or consists of one or more of calcium sulfate, calcium phosphosilicate, sodium phosphate, calcium aluminate, calcium phosphate, hydroxyapatite, partially or fully demineralized bone, or mineralized bone.

The optional particulate material, when present, may comprise any one or more of the materials listed in the embodiments above. In one embodiment, the particulate material, if present in the composition, does not comprise calcium carbonate. In one embodiment, the particulate material may be polymeric, such as a particulated polyurethane.

In embodiments, an optional particulate material is present in an amount of from about 0.01% to about 10% by weight of the composition. In embodiments, the optional particulate material is present in an amount of 0.10% to 10%, 1% to 10%, or 5% to 10%. In other embodiments, the particulate material is present in an amount of from about 10% to about 20% by weight of the composition, or from about 20% to 30%, about 30% to 40%, about 40% to 50%, about 50% to 60%, about 60% to 70% or about 70% to 80% by weight of the composition.

In one embodiment, the particulate additive material is graphene (available from Applied Graphene Materials and Thomas Swan, Ltd.), a single atomic layer of graphite that is electrically conductive, highly elastic, about 100 times stronger than steel and which may be of value improving the quality of tissue healing and new bone stimulation.

The compositions described here may also optionally comprise one or more “cell openers.” Non-limiting examples include ORTOGEL501 (Goldschmidt) and X-AIR (Specialty Polymers & Services). In certain embodiments, the cell openers are present in an amount of from about 0.1% to 5% by weight of the composition. In one embodiment, the cell openers are present in an amount in of from about 1% to 2% or 1% to 3% by weight of the composition.

The compositions described here may also optionally comprise one or more therapeutic agents. In one embodiment, the one or more therapeutic agents are selected from an anti-cancer agent, an antimicrobial agent, an antibiotic, a local anesthetic or analgesic, a statin, and an anti-inflammatory agent. In one embodiment, the antibiotic is selected from a broad spectrum agent, such as gentamicin, clindamycin, and erythromycin, or a gram positive and gram negative family antibiotic such as an ampicillin and a cephalosporin. Benzylalkonium chloride, iodine, and silver sulfadiazine represent examples of non-antibiotic antimicrobial embodiments. In one embodiment, the local anesthetic or analgesic is selected from lidocaine, bupivacaine, tetracaine, and ropivacaine. In one embodiment, the local anesthetic or analgesic is selected from lidocaine, benzocaine and fentanyl (a potent non-opioid anesthetic). In one embodiment, the one or more anti-inflammatory substances is selected from a non-specific anti-inflammatory such as ibuprofen and aspirin, or a COX-2 specific inhibitor such as rofecoxib and celeboxib.

In embodiments, the compositions described herein may optionally further comprise one or more of a radiopaque agent, e.g., barium sulfate, an antioxidant, e.g., tocopheryl acetate, a colorant, e.g., gentian violet or D&C Violet 2 or D&C Green 6, a steroid, e.g., cortisone, and fatty acid salts or esters. In one embodiment, the antioxidant is selected from IRGANOX 1010 and IRGANOX 1035 (Ciba Geigy), and CYANOX 1790 and CYANOX 2777 (Cytec Industries). In certain embodiments, the antioxidant is present in an amount of from about 0.01% to about 5.0% by weight of the composition. In one embodiment, the compositions further comprise one or more growth factors, for example BMP-2, BMP-7, PDGF, EGF, etc.

In certain embodiments, the compositions described here contain no added water. In some embodiments, the compositions are anhydrous. In certain embodiments where there is no added water, water may nevertheless be present in small amounts. For example, certain commercially-available polyols comprise a mixture of the polyol and a small amount of water. In addition, certain optional particulate materials as described herein, such as calcium carbonate may comprise bound water. Formulating the compositions in an atmosphere that contains moisture may also result in the incorporation of water into the compositions. In certain embodiments of the present invention, the compositions are prepared under a dry nitrogen gas purge that comprises a desired minimal amount of moisture, thereby controlling the water content of the compositions. In other embodiments, water may be added to the compositions during the process of their formation from the component parts. In other embodiments, the compositions are prepared under essentially water-free conditions with anhydrous components such that the resulting compositions are essentially anhydrous.

In certain embodiments, water is present in the compositions being made in an amount from about 0.01% to about 3% by weight of the composition. In certain embodiments, water is present in an amount of from about 0.05% to 1%, from about 0.05% to 1.5%, from about 0.1% to 1%, from about 0.1% to 1,5%, from about 0,1% to 2%, from about 1% to 2%, or from about 2% to 3%.

### Lyophilized Sponge Based Materials

The present disclosure provides a solid, lyophilized sponge-based surgical adhesive material having superior properties compared to the aqueous adhesives of the prior art. For example, the lyophilized sponge-based surgical adhesive materials provided herein are physically and chemically more stable, due to their solid form, than liquid adhesives or their precursors. In addition, the solid materials described here are readily sterilized using radiation sterilization.

In embodiments, the disclosure provides a solid, lyophilized sponge-based surgical adhesive material comprising lyophilized sponges, films prepared from lyophilized sponges by compression after humidification, and powders prepared from lyophilized sponges by grinding, milling or micronizing the lyophilized sponges. Unlike liquid adhesives, the solid materials described here do not require any mixing of components just prior to use and due to their solid structure can provide higher concentrations of the active adhesive than would be possible in dilute liquid compositions. Lyophilized sponges are inverse replicates of ice crystals formed by first freezing aqueous solutions or dispersions of polymers and then removing the ice crystals by sublimation under high vacuum. Such sponges exhibit an interconnected pore structure. Lyophilized sponges can be prepared, for example, from materials such as proteins, (e.g. collagen, gelatin), carbohydrates, (e.g., pectin, glycogen, alginic acid and its salts, hyaluronic acid), polymers such as polyvinylpyrrolidone, sodium carboxymethylcellulose, chitosan, and polyacrylic acid salts.

In embodiments, one or more biocompatible, water-soluble polymers is added to the component solutions before lyophilization to ensure the formation of porous, sponge-like structures. The component solutions will generally be comprised primarily of water and may optionally contain a swelling or dispersing agent, a volatile water soluble alcohol, and mixtures thereof. Examples of such polymers that may be added include polyvinylpyrrolidone, sodium carboxymethylcellulose, gelatin, soluble or dispersed collagen fibrils, sodium alginate, sodium hyaluronate, and the like. The porosity of the open-cell sponges thus formed can be controlled by adjusting the lyophilization conditions, e.g., freezing and heating rates. Sponge crystal structure uniformity can be controlled by the addition of small amounts (e.g., 0.1-5%) of biocompatible, volatile, water-miscible liquids, such as ethanol, prior to lyophilization.

In embodiments, the solid, lyophilized sponge-based surgical adhesive material comprises a compressed stack of layers of a porous solid material consisting of, or prepared from, lyophilized sponges. In embodiments, the compressed stack of layers is produced from powders prepared from lyophilized sponges by grinding, milling or micronizing, and compressing the combined powders into a unitary, wafer-like structure under elevated pressure. The so-prepared powders may be sieved one or more times to provide a narrower particle size distribution for improving product uniformity. In accordance with these embodiments, each powder contains reactive components in suitable ratios such that when they are combined and wetted they react to form the finished adhesive. Thus, the resulting individual powders, when combined in the proper ratio and compressed, results in a composite which, when reconstituted with saline or body fluid, forms an adhesive capable of coapting tissue.

### Multi-Putty Settable Adhesives Using Lyophilized Sponge Based Materials

In embodiments, the disclosure further provides a solid surgical adhesive material in the form of a multi-putty composition comprising at least two separate, individual reactive putties. In embodiments, the putties are prepared by mixing a micronized, lyophilized sponge powder with an inert dispersant. The inert dispersant may be a viscous aqueous buffer, for example, an aqueous solution containing a thickening agent such as hypromellose (hydroxypropylmethylcellulose), sodium carboxymethyl cellulose, polyvinylpyrrolidone, or combinations thereof. In embodiments, the inert dispersant may comprise, for example, a poly(alkyleneoxide) glycol or its mono or diester, glycerol, a poloxamer, or a mixture of any of the foregoing. In embodiments, the first putty comprises a suspension of particles from a ground, lyophilized sponge containing a polyanionic polymer salt and the second putty comprises a suspension of particles from a ground, lyophilized sponge containing a polycationic salt such that when the two putties are combined together with a polyvalent metallic salt, such as calcium chloride, they form a coacervate adhesive that cures into a hardened state. In embodiments, the metallic salt can be introduced, for example, in the form of a third putty, or in the form of a powder or solution that is mixed together with the first and second putties of the composition.

In embodiments, an individual putty of the multi-putty adhesive composition consists of a radiation-sterilized, anhydrous, settable surgical adhesive comprising a biocompatible ground, milled or micronized lyophilized, crosslinkable polyanionic salt solution component, a biocompatible ground, milled or micronized lyophilized, crosslinkable polycationic salt solution component and a polyvalent aqueous salt solution optionally containing a biocompatible water soluble polymer, e.g., polyvinylpyrrolidone and/or sodium carboxymethylcellulose component, as a thickening agent.

### Multi-Putty Settable Surgical Materials With Reactive Inorganic Minerals/Inorganic Phosphates

The present disclosure also provides PEG-based multi-putty adhesive compositions. In embodiments, the uncombined putties are water soluble or dispersable putties.

In embodiments, the PEG-based multi-putty adhesive composition comprises two separate, individual reactive putties. In one embodiment, the first putty comprises a mixture of PEG monostearate and PEG containing tetracalcium phosphate, phosphoserine and dry buffer-producing powder and the second putty comprises a mixture of PEG monostearate and PEG containing fibrous or powdered absorbable polyester polymer, e.g. PGA or fibrous or powdered nonabsorbable polyester polymer, e.g. polyethyleneterephthalate. When the separate reactive putties are combined into a homogenous mass and hydrated, e.g., with saline and/or by placing on wet tissue, the resulting composition forms an adhesive that hardens into a cement.

In another embodiment, the first putty is acidic and comprises polyethylene glycol monostearate and polyethylene glycol that contains an acidic phosphate salt, such as monocalcium phosphate monohydrate, an acidic pH buffering agent, such as citric acid, an optional humectant, such as glycerol and a synthetic polymer thickening agent, such as polyvinylpyrrolidone and/or sodium carboxymethyl cellulose and/or hypromellose. The second putty is basic and contains water, a basic calcium phosphate, such as tetracalcium phosphate, a surfactant, such as polysorbate 80 and, optionally, a thickening agent, such as polyvinylpyrrolidone and/or sodium carboxymethyl cellulose and/or hypromellose. Either or both putties may optionally contain an osteoconductive ceramic, such as hydroxyapatite. When the separate reactive putties are combined into a homogenous mass and hydrated with saline or placed on wet tissue, the resulting composition forms an adhesive that hardens into a cement.

The disclosure also provides a general method for avoiding the mixing of aqueous components with dry reactants to form a settable composition. In embodiments, the solid reactants are separated into two groups that remain stable if kept anhydrous, Group 1, for example, may contain putty-like dispersions of powdered tetracalcium phosphate, phosphoserine and a buffer precursor, such as a dry phosphate or carbonate mixture that, when dissolved, will provide a pH of 7-8. The powders may be dispersed in anhydrous PEG stearate/polyalkylene oxide mixtures. Group 2 may contain PEG stearate/polyalkylene oxide putty-like dispersions of fibers or powder prepared from an absorbable polyester, such as polyglycolic acid or a nonabsorbable polyester, such as polyethyleneterephthalate, depending upon whether the adhesive-cement is designed to be absorbable or nonabsorbable in the body after implantation. When the two putties are mixed and sufficiently hydrated, a settable, initially adhesive cement is formed.

The present disclosure also provides a multi-putty settable surgical adhesive composition comprising two separate, individual reactive putties, one putty comprising tetracalcium phosphate powder in an aqueous vehicle and the second putty comprising phosphoserine and polyester in a non-aqueous vehicle. In embodiments, the aqueous vehicle is any water-based vehicle. In embodiments, the aqueous vehicle is saline, or other buffered salt solution, such as phosphate buffered saline. In embodiments, the non-aqueous vehicle is PEG or a fatty acid ester of PEG.

Lally, et al., describe a hemostatic biomaterial (US 2012/0308552) based on combining a mixture of dry powders with saline to form a bone-hemostatic, phosphate-based aqueous slurry. Preparing such a slurry is inconvenient and the present disclosure, involving combining putties in which the components are dispersed, avoids the inconvenient powder/saline preparation and also provides a putty-like composition that is more easily applied and is more adherent to bone than is an aqueous slurry.

### Multi-Putty Cements Based on Poly (Methyl Methacrylate) (PMMA)

The present disclosure also provides multi-putty surgical adhesive compositions consisting of at least two separate, individual reactive putties wherein the reactive components comprise liquid acrylate and methacrylate monomers and powdered polyacrylate and polymethacrylate polymers. In embodiments, the multi-putty adhesive composition comprises two separate, individual reactive putties. In one embodiment, the first putty comprises one or more liquid acrylate and methacrylate monomers and polyacrylate and polymethacrylate polymers with a solubilizing vehicle such as ethyl acetate or N-methylpyrrolidone, to form a putty. The second putty comprises a free radical source, such as benzoyl peroxide, or an ionic source, such as ferric acetate, and one or more powdered polyacrylate and polymethacrylate polymers dissolved or suspended in an inert liquid vehicle, such as ethyl acetate or N-methylpyrrolidone, to form a putty. When the two putties are combined, polymerization of the monomers in the first putty is initiated by the free radical or ionic source in the second putty which avoids the more difficult mixing together of solids and liquids.

### Multi-Putty Settable Bioplastic Surgical Material

U.S. Pat. No. 8,529,960 (Campbell) describes blood-derived plastic articles prepared from whole blood plasma that can be useful for wound repair and tissue grafts. The present disclosure provides an improved composition based on the plasma-based putty described by Campbell, In embodiments, the present disclosure provides a multi-putty settable surgical composition comprising two separate individual reactive putties, a first putty comprising an incubated mixture of a polyol and freeze-dried clotted blood plasma and a second putty comprising a polyisocyanate terminated prepolymer. The first putty can be made, for example, by clotting human plasma (e.g., with calcium chloride) and lyophilizing the clotted material together with its serum to a water content of about 8%, followed by milling or grinding and sieving (150 microns) to produce particles which are combined with glycerol and incubated for about 21 hours in a closed container. The second putty comprises an absorbable polyisocyanate-terminated prepolymer. Combining the two putties together forms an absorbable, adhesive, settable bioplastic material because the hydroxyl and amino groups present in the first putty react with the polyisocyanates in the second putty to form an absorbable polyurethane network.

If desired, the first or second putty, or both, can be made porous, for example, by adding ammonium acetate crystals (which are later removed by sublimation) or dextrose crystals (which are later removed by aqueous dissolution). The putty can optionally be crosslinked with, for example, glutaraldehyde or, preferably, with the less toxic genipin.

Biological response modifiers, e.g., growth factors, and drugs such as antimicrobials, hormones, anti-inflammatory agents, anti-neoplastic agents, etc., also optionally may be added to either the first or second putty.

### Multi-Putty Settable Surgical Materials Based on Epoxides

The disclosure also provides multi-putty settable surgical materials based on epoxides as the reactive group. In one embodiment, the polymer system is based upon reactive epoxide groups which form catalytic homopolymerizations or, alternatively, copolymers by reaction with curatives or hardeners such as polyfunctional amines, acids, phenols, alcohols, anhydrides or thiols and combinations thereof. As with PMMA, curing of epoxy resins is exothermic and should be controlled to avoid adverse thermal effects on tissue. Aliphatic and cycloaliphatic epoxides react more slowly than do aromatics and exhibit lower exotherms.

While uncured epoxy resins are potentially irritating and sensitizing, cured polymers are essentially non-toxic and have been used, for example, to encapsulate implantable cardiac pacemakers (U.S. Pat. Nos. 3,700,628, 3,924,640). Earlier reports of cured epoxy carcinogenicity now are attributed to the presence of carcinogenic epichlorohydrin which is no longer used for preparing most epoxy resins.

In one embodiment, an epoxy resin such as triglycidal-p-aminophenol is liquid at room temperature and has relatively high reactivity. It may be cured with a cycloaliphatic amine catalyst such as dicyclohexyl amine. The advantage of this embodiment over prior art is that both the liquid epoxy resin and the curing components are converted to putty-like consistencies by the addition of micronized solid fillers such as phosphate salts and/or ceramics or by non-reactive, finely ground polymers such as PVP or ground previously formed epoxy-based polymers. Aseptically hand kneading such sterile putties together provides a surgically useful initially adhesive cement.

Copolymers made with a hydrolysable polyol such as, e.g., the diglycolate ester of 1,3-propane diol in a stochiometrically correct ratio with the expoy resin will provide an absorbable version of the surgical device. The epoxide resin backbone also can be made with hydrolysable linkages such as esters, e.g., glycidyl-CH₂—CH₂—CO₂—CH₂—CH₂-glycidyl, to form absorbable polymers which have been further enhanced regarding absorbability be combining hydrolysable epoxy resins with hydrolysable polyols.

In one embodiment, the disclosure provides a multi-putty settable surgical composition comprising two separate, individual reactive putties, a first putty comprising an aliphatic and/or aromatic polyglycidyl epoxy resin of sufficient molecular weight to provide a putty-like consistency and a second putty containing an optional thickener and a polyfunctional primary and/or secondary amine, polyfunctional anhydride, polyfunctional phenol or polyfunctional In one embodiment, the disclosure provides a surgical composition formed by combining the two separate individual reactive putties into a single homogenous mass, wherein the composition is absorbable or nonabsorbable.

The following non-limiting examples are intended to further illustrate and exemplify the multi-putty compositions described here.

### EXAMPLES

The following table provides examples of multi-putty settable surgical materials based on epoxides (1,2); bioplastics (3); PMMA (4); sponges (5); phosphoserine (6); and PEG (7).

| | | | | Component | |
|---|---|---|---|---|---|
| | | Reactive components | Additive | Putty | Combined Putty |
| # | Putty | (wt %) | (wt %) | Observations | Observations |
| 1 | A | Putty comprising 60-80% | None | | While reacting, |
| | | of trigtycidal-p-amino | | | the combined |
| | | phenol and 20-40% of a | | | putties may be |
| | | thickener comprised of | | | used as a |
| | | micronized polyvinyl | | | nonabsorbable |
| | | pyrrolidone or of | | | cement for hard |
| | | micronized calcium | | | tissue such as |
| | | phosphate | | | fractured or as a |
| | B | Putty comprising 60-80% | Optional 5% d,l- | | hemostat to |
| | | dicyclohexyl amine and | tocopheryl acetate. | | control bleeding |
| | | 20-40% of a thickener | Optional drugs such as | | bone. |
| | | comprising micronized | 2% antimicrobial | | |
| | | hydroxyapatite | agents Optional | | |
| | | | porogens such as 10% | | |
| | | | fructose crystals. | | |
| 2 | A | Same as 1A | Same as 1A | | Composition |
| | B | Putty comprising 60-80% | Optional porogen such | | provides a |
| | | of a comonomer of the | as 10% sugar fibers | | resorbable epoxy |
| | | diglycidal ester of | (cotton candy) | | polymer with |
| | | ethylene glycol-beta- | | | hydrolysable ester |
| | | hydroxypropionate and | | | linkages in Putty B |
| | | 20-40% of a thickener | | | |
| | | comprised of micronized | | | |
| | | calcium phosphate | | | |
| 3 | A | 40-60% of a putty | Optional 2.0% of an | | The combined |
| | | comprised of 80 parts of | anesthetic, e.g., | | putties form an |
| | | powdered (milled), | lidocaine | | absorbable |
| | | lyophilized mixture of | Optional clot | | cement or sealant |
| | | clotted human plasma | crosslinking agent - | | through the |
| | | and its serum component | 2% genipin | | prepolymer |
| | | that was incubated in 20 | | | isocyanate |
| | | parts of glycerol at 45° C. | | | reacting with the |
| | | for 24 hours | | | glycerol from |
| | B | 60-40% of a putty | | | Putty A to form a |
| | | comprised of an | | | polyurethane and |
| | | isocyanate-terminated | | | with amino |
| | | block polyalkylene oxide | | | groups, from the |
| | | glycol prepolymer | | | tissue being |
| | | | | | cemented or |
| | | | | | sealed, to form a |
| | | | | | polyureaurethane. |
| 4 | A | Putty comprising 50% | Optional 0.25% D&C | Each putty can | Useful as a hip |
| | | methyl methacrylate, | Green 6 dye in Putty A | alternatively, be | prothesis femoral |
| | | 20% methyl acrylate and | to add color | hand mixed using | stem anchor. |
| | | 10% polymethyl acrylate | Optional 5% osteogenic | double gloves to | |
| | | and 10% | ceramic to reinforce | insulate against | |
| | | polymethylmethacrylate | the polymer and | exothermic heat | |
| | | as thickener polymers | stimulate bone growth | | |
| | | dissolved in 10% N- | | | |
| | | methylpyrrolidone | | | |
| | B | Putty comprising 5% | | | |
| | | benzoyl peroxide, 10% | | | |
| | | polymethyl acrylate and | | | |
| | | 10% polymethyl | | | |
| | | methacrylate as | | | |
| | | thickeners dissolved in | | | |
| | | N-methylpyrrolidone | | | |
| 5 | A | Lyophilized 7% sodium | None | Each film can be | Alternatively, the |
| | | carboxymethyl cellulose | Option to sodium CMC | individually | A and B compressed |
| | | sponge finely ground | is 7% poty 1-DOPA | suspended in | sponges can be |
| | | particles containing | | waler to form | combined together |
| | | micronized 5% | | putty-like | in water to form |
| | | phosphate buffer dry | | suspensions that | the complex |
| | | precursor particles | | when mixed, form | coacervate cement. |
| | | compressed into a thin | | adherent complex | |
| | | particulate film | | coacervates that | |
| | | | | cure to form tissue | |
| | | | | cements even | |
| | | | | under aqueous, | |
| | | | | tissue-like | |
| | | | | environments. | |
| | B | Lyophilized 7% chitosan | None | | |
| | | hydrochloride sponge | Option to chitosan | | |
| | | finely ground particles | hydrochloride is 7% | | |
| | | containing 5% phosphate | poly(coglycine/lysine) | | |
| | | buffer dry precursor | hydrochloride in a 2:1 | | |
| | | particles and 2% finely | ratio | | |
| | | ground dry calcium | | | |
| | | chloride compressed into | | | |
| | | a thin particulate film | | | |
| 6 | A | Putty comprised of 35% | | Hand moldable | |
| | | polyethyleneglycol | | putty | |
| | | monostearate, 10% | | | |
| | | polyethylene glycol, | | | |
| | | 30% tetracalcium | | | |
| | | phosphate, 20% | | | |
| | | phosphoserine and 5% | | | |
| | | dry phosphate buffer | | | |
| | | precursor powder | | | |
| | B | Putty comprised of 35% | Optional: 0.2% D&C | Hand moldable | |
| | | polyethyleneglycol | Violet 2 | putty | |
| | | monostearate, 30% | | | |
| | | polyethylene glycol, 30% | | | |
| | | micronized polyglycolic | | | |
| | | acid (for absorbable | | | |
| | | cements) or, 30% | | | |
| | | micronized | | | |
| | | polyethyleneterephthalate | | | |
| | | (for nonabsorbable | | | |
| | | cements), 5% dry | | | |
| | | phosphate buffer | | | |
| | | precursor powder | | | |
| 7 | A | Putty comprising 30% | | Hand moldable | |
| | | finely powdered KH₂PO₄ | | putty | |
| | | (monopotassium | | | |
| | | phosphate), 15% finely | | | |
| | | powdered calcined MgO | | | |
| | | (magnesium oxide), 45% | | | |
| | | polyethyleneglycol | | | |
| | | monostearate, 10% | | | |
| | | triacetin | | | |
| | B | Putty comprising 20% | Optional: 0.2% D&C | Hand moldable | |
| | | finely powdered | Violet 2 | putty | |
| | | hydroxyapatite, 20% | | | |
| | | finely powdered sucrose, | | | |
| | | 50% polyethyleneglycol | | | |
| | | monostearste, 10% | | | |
| | | triacetin | | | |

### Equivalents

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A settable surgical adhesive composition comprising a compressed stack of dry layers of lyophilized sponges, or particles or films of lyophilized sponges, or any combination thereof, wherein the compressed stack of dry layers, when wetted with an aqueous solution, form an adhesive composition.

2. The settable surgical adhesive composition of claim 1, in which a first layer contains a polyanionic salt, a second layer contains a polycationic salt, and a third layer contains a polyvalent metallic salt, each individual layer containing an optional biocompatible cryoprotective agent and an optional dry buffer-producing powder.

3. A multi-putty settable surgical adhesive composition comprising two separate, individual reactive putties, a first putty comprising particles from a ground lyophilized sponge containing a polyanionic polymer salt and a second putty comprising particles from a ground lyophilized sponge containing a polycationic polymer salt wherein, when the two putties are combined together with a polyvalent metallic salt and, when wetted with an aqueous solution, form an adhesive that cures into a hardened state.

4. A multi-putty settable surgical adhesive composition comprising two separate, individual reactive putties, a first putty comprising a mixture of polyethylene glycol (PEG) monostearate and PEG containing tetracalcium phosphate, phosphoserine and dry buffer-producing powder and a second putty comprising a mixture of PEG monostearate and PEG containing fibrous or powdered absorbable polymer containing backbone ester groups or fibrous or powdered nonabsorbable polymer containing backbone ester groups.

5. The surgical composition formed by combining the two separate individual reactive putties of claim 4 into a single putty.

6. A multi-putty settable surgical adhesive composition comprising two separate, individual reactive putties, a first putty being acidic and comprising an alkylene oxide glycol mono-fatty acid ester, an alkylene oxide glycol, an acidic phosphate salt, an acidic pH buffering agent, an optional humectant and an optional poloxamer and a second putty being basic and comprising an alkylene oxide glycol mono fatty acid ester, an alkylene oxide glycol, a basic calcium phosphate salt, a surfactant and, optionally, osteoconductive and/or osteoinductive agents.

7. The surgical composition formed by combining the two separate individual reactive putties of claim 6 into a single putty.

8. A multi-putty settable surgical adhesive composition comprising two separate, individual reactive putties, a first putty comprising one or more basic calcium phosphate salts, a polyol, a polyol-terminated polyurethane prepolymer and an optional osteoactive ceramic and a second putty comprising an acidic calcium phosphate salt, a polyisocyanate, an isocyanate-terminated prepolymer, and an optional osteoactive ceramic.

9. The surgical composition formed by combining the two separate individual reactive putties of claim 8 into a single putty.

10. A multi-putty settable surgical adhesive composition comprising two separate, individual reactive putties, a first putty comprising an aliphatic or aromatic polyisocyanate, an osteoactive calcium phosphate, magnesium oxide, dibasic potassium phosphate, tetracalcium phosphate and a viscous isocyanate-terminated polyurethane prepolymer, and a second putty comprising phosphoserine, one or more polyols, BMP and/or DBM, optionally N-methyl pyrrolidone, an osteoactive calcium phosphate and a viscous, hydroxyl-terminated polyurethane prepolymer.

11. The surgical composition formed by combining the two separate individual reactive putties of claim 10 into a single putty.

12. A multi-putty settable surgical composition comprising two separate, individual reactive putties, a first putty comprising powdered monobasic potassium phosphate and powdered calcined magnesium oxide dispersed in an anhydrous mixture of PEG diacetate and monoacetyl PEG stearate, optionally containing an osteoactive calcium phosphate; and a second putty comprising a dispersion of powdered beta-tricalcium phosphate and powdered sucrose in an anhydrous mixture of PEG diacetate and monoacetyl PEG stearate, optionally containing an osteoactive ceramic.

13. The surgical composition formed by combining the two separate individual reactive putties of claim 12 into a single putty.

14. A multi-putty settable surgical composition comprising two separate, individual reactive putties, a first putty comprising an aliphatic and/or aromatic polyglycidyl epoxy resin of sufficient molecular weight to provide a putty-like consistency and a second putty comprising an optional thickener and a tertiary amine catalyst to form a homopolymer through catalytic homopolymerization.

15. The surgical composition formed by combining the two separate individual reactive putties of claim 14 into a single putty, wherein the composition is absorbable or nonabsorbable.

16. A multi-putty settable surgical composition comprising two separate, individual reactive putties, a first putty comprising an aliphatic and/or aromatic polyglycidyl epoxy resin of sufficient molecular weight to provide a putty-like consistency and a second putty containing an optional thickener and a polyfunctional primary and/or secondary amine, polyfunctional anhydride, polyfunctional phenol or polyfunctional thiol.

17. The surgical composition formed by combining the two separate individual reactive putties of claim 16 into a single putty, wherein the composition is absorbable or nonabsorbable.

18. A multi-putty settable surgical composition comprising two separate individual reactive putties, a first putty comprising liquid acrylate and/or methacrylate monomers and acrylate and/or methacrylate polymers and a second putty comprising a free radical catalyst precursor or an ionic catalyst precursor in a biocompatible organic solvent.

19. The surgical composition of claim 18, wherein the biocompatible organic solvent is selected from ethyl acetate or an N-alkylpyrrolidone.

20. The surgical composition of claim 18, wherein the free radical catalyst precursor is benzoyl peroxide.

21. The surgical composition of claim 18, wherein the ionic catalyst precursor is ferric acetate.

22. The surgical composition of claim 18, wherein the biocompatible organic solvent is ethyl acetate or an N-alkylpyrrolidone.

23. The surgical composition formed by combining the two separate individual reactive putties of claim 18 into a single putty.

24. A multi-putty settable surgical composition comprising two separate individual reactive putties, a first putty comprising an incubated mixture of a polyol and freeze-dried clotted blood plasma and a second putty comprising a polyisocyanate terminated prepolymer.

25. The surgical composition formed by combining the two separate individual reactive putties of claim 24 into a single putty.

26. A multi-putty settable surgical adhesive composition comprising two separate, individual reactive putties, one putty comprising tetracalcium phosphate powder in an aqueous vehicle and the second putty comprising phosphoserine and polyester in a non-aqueous vehicle.

27. The composition of any of claims 1-26, wherein the composition is sterile.
